(19) United States
(12) Patent Application Publication
(10) Pub. No.: US 2005/0175534 A1
(43) Pub. Date: Aug. 11, 2005

(54) Method of synthesizing compounds having a phosphorus-fluorine-18 bond

(76) Inventors: Michael J. Adam, Vancouver (CA); Andrei R. Studenov, Vancouver (CA); John S. Wilson, Edmonton (CA)

Correspondence Address: HARNESS, DICKEY & PIERCE, P.L.C., P.O. BOX 8910, RESTON, VA, 20195, US

(21) Appl. No.: 11/049,951

(22) Filed: Feb. 4, 2005

Related U.S. Application Data
(60) Provisional application No. 60/541,945, filed on Feb. 6, 2004.

(51) Int. Cl. A61K 51/00

(52) U.S. Cl. 424/1.11; 534/11

(57) ABSTRACT

Provided are methods for synthesizing radiotracers, including no carrier added (n.c.a.) tracers suitable as PET scan tracers that contain a ¹⁸F—P bond. The radiolabeled products may be synthesized from precursors including phosphorus(V) and/or phosphorus (III) atoms bound to a suitable leaving group or group(s). For example, precursors incorporating a nitrophenoxy leaving group bound to a phosphorus(V) atom tend to exhibit an acceptable combination of stability and reactivity and also tend to allow the precursor compound(s) to be separated more easily from the ¹⁸F-labeled products. The methods disclosed herein for producing radiolabeled compounds incorporating a P—¹⁸F chemistry may particularly useful radiolabeling oligonucleotides, phospholipids, phosphorilated proteins, sugars and steroids for use as PET radiotracer compounds for medical imaging applications.

### PRIORITY CLAIM

This Application claims priority from U.S. Provisional Application No. 60/541,945, filed Feb. 6, 2004, the contents of which are hereby incorporated in its entirety by reference.

### BACKGROUND OF THE INVENTION

1. Field of the Invention

The present invention relates to a method for synthesizing compounds containing a ¹⁸F—P bond in which the radiolabeling is achieved by nucleophilic substitution of an appropriate leaving group attached to the phosphorus, the phosphorus having an oxidation state of P(V) or P(III), by an ¹⁸F⁻ ion in a polar aprotic solvent. Exemplary leaving groups include fluoride, chloride and 4-nitrophenoxy groups. This method of synthesizing radiolabeled compounds will be especially useful for synthesizing PET radiotracers including radiolabeled oligonucleotides, phospholipids, phosphorilated proteins, sugars and steroids.

2. Description of the Related Art

Phosphorus containing compounds are found through biological systems as, for example, monomers such a nucleotides, phospholipids and phosphorylated carbohydrates and more complex oligomers and polymers such as DNA and RNA. Compounds containing a phosphorus-fluorine bond have been used in biological studies, as reported by Anderson et al. in Biochemistry 1997; 36: 2586-2594, and as poisons, as reported by Green et al., in J. Chem. Soc. Abst. 1958: 1583-1587; and by Ashani et al., in Biochem Pharmacol 1998; 55: 159-168. Extensive literature exists on the chemistry of the P—F bond and special attention has been paid to the fluorinated oligonucleotide analogs as reported by Bollmark et al., in Nucleosides & Nucleotides 1998; 17: 663-680.

None of these compounds, however, has been labeled using a no carrier added (n.c.a.) positron emitter fluorine-18 (half-life 110 min) or used as a PET radiotracer. Almost all of the suggested PET radiotracers contain a ¹⁸F—C bond as reported by Iwata, R., Division of Radiopharmaceutical Chemistry, CYRIC TOHOKU University Website http://kakuyaku.cyric.tohoku.acjp; and Fowler et al., in Semin. Nucl. Med. 2003; 33: 14-27.

One exception to the 18F—C bond are proposed radiotracers containing an ¹⁸F—Si bond, but these compounds are expected to suitable for a limited range of applications as reported by Walsh et al., in J. Labelled Cpd Radiopharm. 1999; 42: S1-S3. A synthesis of sodium fluorophosphate double labeled with ¹⁸F and ³²P, accomplished via the high temperature (800° C.) solid phase reaction of the carrier added [¹⁸F]NaF with carrier added [³²P]NaPO₃, was described in the earlier 1960's by Ericson in Int'l. J Appl. Radiat. Isot. 1961; 10: 177-180.

One concern with respect to the development of any new radiotracer with ¹⁸F-element bond in which the other bonding element is not carbon is the stability of the resulting compound under the physiological conditions associated with the intended use of the radiotracer. Although some oligonucleotide fluorophosphodiesters are hydrolytically unstable for in vivo use, as reported by Misiura et al., in Med. Chem. 2001; 9: 1525-1532, a negative charge on the oxygen adjacent the P—F bond tends to produce fluorophosphomonoesters having sufficient stability. For example, an AZT (3′-azido-3′-deoxythymidine, RETROVIR®) analog containing a P—F bond was sufficiently stable to be considered as a potential therapeutic drug as reported by Dyatkina et al., in Nucleosides & Nucleotides 1994; 13: 325-337 and by Egron et al., in Bioorg. Chem. 2001; 29: 333-344 (“Egron”). Storing this compound in an aqueous solution for three days did not, apparently, result in any significant decomposition. As reported by Egron, the most “aggressive” bacterial media studied decomposed the AZT analog at a rate resulting in an effective half-life of about 2 hours.

### BRIEF SUMMARY OF THE INVENTION

Disclosed are methods for manufacturing ¹⁸F radiolabeled phosphorus-containing compounds. A first method includes preparing an anhydrous solution of a phosphorus-containing precursor compound, the precursor compound including at least one phosphorus atom bonded to a leaving group; introducing an activated 18F fluoride into the anhydrous solution to form a reaction mixture; and maintaining the reaction mixture at a reaction temperature and for a reaction time sufficient to cause a first percentage of the ¹⁸F to replace the leaving group through nucleophilic substitution to form a radiolabeled compound.

The method may also include the step of separating the radiolabeled compound from the reaction mixture. Each of the precursor compounds will include a leaving group bound to a P(III) or P(V) phosphorus atom that, during the synthesis reaction, will be replaced by one of the activated [¹⁸F]F-ions in the solution to produce the radiolabeled product. Exemplary leaving groups include chlorine, fluorine, nitrophenyl and nitrophenoxyl. Depending on the components of the reaction mixture, the reaction temperature may range from about 25° C. to about 80° C., but will typically reflect a compromise between increasing the reaction rate and degrading one or more of the organic components of the reaction mixture. Similarly, the reaction times may be relatively brief, on the order of several minutes, or may extend for hours or even days.

A range of precursor compounds may be utilized in the disclosed radiolabeling method including, for example, nucleotides, oligonucleotides, phospholipids, phosphorilated proteins, phosphorilated sugars and steroids that exhibit both a carbon-phosphorus bond and a phosphorus-leaving group bond. Similarly, a variety of methods may be used to separate the ¹⁸F radiolabeled phosphorus-containing compounds from the reaction mixture. As described in more detail below, thin layer chromatography (“TLC”) using a variety of substrates and solvents may be utilized to separate the various components of the reaction mixture. Once the various compounds, or at least the desired product compound, have been sufficiently separated within the chromatography medium or substrate, the portion of the chromatography substrate containing the desired compound may be eluted to obtain an eluate product solution containing the radiolabeled compound.

The activated ¹⁸F fluoride may be prepared by combining an aqueous [¹⁸F]F-solution with a solution of tetrabutylammonium carbonate in acetonitrile to form an activated fluoride solution and then evaporating the solvent from the activated fluoride solution to obtain an activated ¹⁸F fluoride compound. Another method of preparing the activated ¹⁸F fluoride includes combining an aqueous [¹⁸F]F-solution with a solution of 4,7,13,16,21,24-hexaoxa-1,10-diazabicyclo[8.8.8]hexacosane and K₂CO₃ in a acetonitrile:water solvent to form an activated fluoride solution and then evaporating the solvent from the activated fluoride solution to obtain an activated ¹⁸F fluoride compound.

In some instances, the precursor compound may include a phosphorus atom having an oxidation state of (III). In such instances, it may be beneficial to incorporate an additional step into the process in order to increase the oxidation state of the phosphorus atom to (V). Both oxidation and sulfurization methods may be utilized to achieve this result and thereby generate an intermediate precursor for combination with the activated fluoride that will tend to produce a more stabile radiolabeled product.

### DETAILED DESCRIPTION OF EXEMPLARY EMBODIMENTS

In connection with the exemplary embodiments detailed below, a 0.5 M solution of methanolic sodium methoxide was prepared by adding 2.29 g of freshly cut sodium to 200 mL of anhydrous methanol. ¹H NMR spectra were recorded with Brucker 300 MHz or 500 MHz while ³¹P NMR, with 200 MHz NMR spectrometers. Preparative thin layer chromatography (TLC) was from Analtech (Newark, Del.), Silica GF, 20×20 cm, 1 mm thick. The chemical and radiochemical purity were determined by radio TLC (using a Macherey-Nagel, G/UV₂₅₄ plastic-back TLC plates, 4×8 cm) and analytical radio high pressure liquid chromatography (HPLC) in the presence of a corresponding, but unlabeled, carrier compound.

Because of the very close chromatographic properties, in some cases, chloride-precursors were substituted for fluoride-reference compounds to increase the separation of the compounds and improve the detection sensitivity. The compounds on the TLC plates were then visualized using UV light, iodine staining and/or ninhydrin treatment as necessary.

An exemplary method for preparing an anhydrous reactive [¹⁸F]F⁻ component utilizes fluoride activated with tetrabutylammonium carbonate. In this method, tetrabutylammonium carbonate (10 μL of 1.5 M solution in acetonitrile prepared as described by Culbert et al., in Applied Radioactive Isotopes, 1995; 46: 887-891, the disclosure of which is incorporated herein, in its entirety, by reference) was combined with an aqueous [18F]F⁻ solution, with the resulting solution being evaporated under argon using an oil bath maintained at a temperature of about 80° C. Additional acetonitrile (3×0.3 mL) was repeatedly added and subsequently evaporated to obtain a substantially anhydrous final ¹⁸F fluoride composition.

Another exemplary method for preparing an anhydrous reactive [¹⁸F]F⁻ component utilizes fluoride activated with KRYPTOFIX®/K₂CO₃. In this method, a solution including KRYPTOFIX 222 (4,7,13,16,21,24-hexaoxa-1,10-diazabicyclo[8.8.8]hexacosane, 10 mg, 27 μmol) and K₂CO₃ (3 mg, 22 μmol) in 1:1 acetonitrile:water (0.2-0.3 mL) was added to the aqueous [18F]F⁻. The resulting solution was evaporated under argon using an oil bath maintained at a temperature of about 100° C). Acetonitrile was then repeatedly added and evaporated (3×0.3 mL) to obtain a substantially anhydrous composition.

### Synthesis Of ¹⁸F—P-Labeled Compounds From Phosphorus(V) Precursors

### Synthesis of [¹⁸F]Dimefox ([¹⁸F]bis(dimethylamido)phosphoric fluoride)(2a)

A solution of bis(dimethylamido)phosphoric chloride (1a) (10 μL, 67 μmol) in acetonitrile (0.4 mL) was combined with the dried [¹⁸F]F⁻ activated with tetrabutylammonium carbonate described above. The resulting solution was maintained at room temperature (approximately 25° C.) for 5 minutes, after which a sample was withdrawn for analysis. The remainder of the solution was then heated to approximately 75° C. and maintained at this temperature for approximately 10 minutes to promote nucleophilic substitution and thereby synthesize the [¹⁸F]bis(dimethylamido)phosphoric fluoride (2a) according to the general reaction (I) as illustrated below.

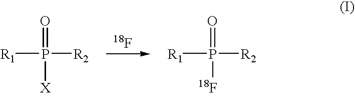

Analysis of the reaction product using by three different radio TLC methods, specifically Silica with 1:1 acetone:hexane eluent, Rf (2a) 0.47, (chloride precursor) 0.55; Alumina-N, 1:1 acetone:hexane, Rf(2a) 0.60; and C18, 30 mM ammonium acetate-40% acetonitrile, Rf (2a) 0.60, confirmed formation of the radiolabeled dimefox (2a). Regions corresponding to the unlabeled dimefox (Riedel-de Haen) and its chloride precursor were visualized by spraying the TLC plates with a freshly prepared 1:2 mixture of concentrated aqueous HCl:2% ninhydrin in acetone followed by heating according to the method outlined by Coha in J. Chromatography, 1968; 34: 558-559. This reference is hereby incorporated, in its entirety, by reference. This treatment produced red-brown spots on the light-red background on the Silica plates and brown spots on the white- and the gray backgrounds provided by the Alumina N and C18 plates respectively. Analysis of the plates indicated conversion of the solubilized [¹⁸F]F⁻ into the [¹⁸F]bis(dimethylamido)phosphoric fluoride (2a) was approximately 96%. The hydrolytic stability of the [¹⁸F]bis(dimethylamido)phosphoric fluoride (2a) was investigated using an aliquot (50 μL) of the reaction mixture mixed with water (50 μL) with a TLC analysis being conducted at the 10 minute and 30 minute time points.

### Synthesis of [¹⁸F]Diphenyl fluorophosphate (2b)

Dimethylformamide (DMF) (0.4 mL) was added to the dried [¹⁸F]F⁻ activated by KRYPTOFIX/K₂CO₃ followed by the addition of diphenyl chlorophosphate (1b) (10 μL, 48 μmol). The solution was heated to about 90° C. and maintained at this temperature for about 10 minutes to promote nucleophilic substitution and thereby synthesize the [¹⁸F]Diphenyl fluorophosphate (2b) according to the general reaction (I) as illustrated above. A small fraction (30 μL) of the reaction mixture was then withdrawn, diluted with ethanol (60 μL) and analyzed using TLC to confirm the presence of the desired radiolabeled product [18F]Diphenyl fluorophosphate (2b). TLC, Silica, 1:2 acetone: hexane, Rf (2b) 0.42; 5% methanol-CH₂Cl₂, Rf (2b) 0.85 (the radiolabeled product was at the same location on the TLC plates as the Cl-precursor used as a reference).

### Synthesis of Diethyl-4-nitrophenylphosphate (1d)

Methanolic sodium methoxide (0.5 M, 1 mL) was added to 4-nitrophenol (69 mg, 0.5 mmol); the bright yellow solution was stirred briefly and then concentrated using vacuum evaporation to obtain an orange residue. The orange residue was then suspended in acetonitrile (10 mL) and re-evaporated to remove residual methanol. Diethylchlorophosphate (0.1 mL, 0.5 mmol) was added to the solution of sodium 4-nitrophenolate in DMF (3 mL), after which the reaction mixture was heated to approximately 80° C. and maintained at this temperature for approximately 15 minutes.

The reaction mixture was then evaporated and the residue was partitioned between ethyl acetate and water. The organic phase was washed with water and brine before it was filtered and evaporated. A portion of the mixture was applied to a preparative TLC [CH₂Cl₂, Rf (1d) 0.45] to confirm the presence of the desired product. The product was eluted from Silica with chloroform:methanol (9:1), and the solvent was evaporated to yield diethyl-4-nitrophenylphosphate 1d (20 mg, 73 μmol, 15% yield) as an oil. Analysis of the product produced the following results: Mass Spectroscopy (MS) (E.I.) M⁺ 275.0560 (51.85%), M+1⁺ 276.0597 (6.25). Formula Weight (FW) 275.0559. ¹H NMR (CDCl₃, 500 MHz) δ 8.24 (2H, dt, 9.0 Hz, 0.7 Hz), 7.37 (2H, dt, 9.0 Hz, 1.0 Hz), 4.26 (4H, m), 1.38 (6H, dt, 7.0 Hz, 1.0 Hz).

### Synthesis of [¹⁸F]Diethyl fluorophosphate (2c)

A solution of diethylchlorophosphate (1c) (10 μL, 70 μmol) in acetonitrile (0.4 mL) was added to the dried [¹⁸F]F⁻ activated by KRYPTOFIX/K₂CO₃. The reaction solution was heated to approximately 70° C. and maintained at this temperature for approximately 5 minutes to promote nucleophilic substitution and thereby synthesize the [18F]Diethyl fluorophosphate (2c) according to the general reaction (I) as illustrated above. The resulting mixture was analyzed for the presence of the desired [¹⁸F]Diethyl fluorophosphate (2c) using the precursor diethylchlorophosphate (1c) as a reference. TLC, Silica, 1% methanol-chloroform, Rf (1c, weak spot upon prolonged iodine staining) 0.45, (2c) 0.54. Conversion of the solubilized [¹⁸F]F⁻ into the product was approximately 35%. In the carrier added experiment, tetrabutylammonium fluoride (20 μL of 1 M solution in THF) was added to the aqueous [¹⁸F]F⁻ (10 μL); CH₃CN (3×0.5 mL) was added and evaporated to leave a dry residue for the reaction with the precursor diethylchlorophosphate (1c). Conversion of the carrier added [¹⁸F]F⁻ into the [¹⁸F]diethyl fluorophosphate (2c) product was approximately 90%.

### Synthesis of [¹⁸F]Diethyl-4-fluorophosphate (2d)

The solution of the precursor diethyl-4-nitrophenylphosphate (1d) (1 mg, 3.6 μmol) in acetonitrile (100 μL) was added to the dried [¹⁸F]F⁻ activated with KRYPTOFIX/K₂CO₃ as described above. The reaction mixture was then heated to a temperature of approximately 75° C. and maintained at that temperature for approximately 15 minutes to promote nucleophilic substitution and thereby synthesize the radiolabeled [¹⁸F]diethyl-4-fluorophosphate (2d) product according to the general reaction (I) as illustrated above. A sample of the post-reaction mixture was analyzed using TLC methods as detailed above and indicated that the conversion of the solubilized [¹⁸F]F⁻ represented by radiolabeled product was approximately 75%. The hydrolytic stability of the product produced in the carrier added reaction, was evaluated with aliquots (50 μL) of the reaction solution that were treated with 10 μL of water or 10 μL of 0.5 M aqueous K₂CO₃. The treated samples were stored for approximately 20 minutes at ambient temperature, approximately 25° C., and then heated to a temperature of approximately 70° C. and maintained at that temperature for approximately 10 minutes before being cooled and evaluated for product degradation.

### Synthesis of Ethyldi(4-nitrophenyl) phosphate (1e)

Methanolic sodium methoxide (0.5 M, 1 mL) was added to 4-nitrophenol (69 mg, 0.5 mmol), and the solution was briefly stirred before concentration in vacuo to obtain a sodium 4-nitrophenoxide product. The recovered sodium 4-nitrophenoxide (orange residue) was then suspended in anhydrous acetonitrile (10 mL) and re-evaporated to remove traces of methanol. The residue was then added to DMF:dioxane (1:1, 6 mL) to which ethyldichlorophosphate (0.1 mL, 0.5 mmol) was added to form a reaction mixture. After about 0.5 hour the reaction mixture was evaporated to dryness, after which the solid obtained by evaporation was partitioned between ethyl acetate and water. The organic phase was washed with water and brine, filtered, and evaporated. The resulting product was purified using preparative TLC, Silica, CH₂Cl₂, Rf 0.35. The desired zone of the TLC plate was eluted with methanol:chloroform 1:9 after which solvent was evaporated from the eluate to obtain a solid residue. This residue was then recrystallized using cyclohexane/acetone to obtain approximately 20 mg of the desired ethyldi(4-nitrophenyl) phosphate (1e), 54 μmol, 22% yield) as fine white needles. Analysis of the product produced the following results: ³¹p NMR (CDCl₃) δ-12.351 ppm ¹H NMR (CDC1₃, 500 MHz) δ,8.27 (4H, dt, 9.2 Hz,0.7 Hz),7.41 (4 H, dt, 9.2Hz, 1.0 Hz), 4.10 (2H, dq, 7.0 Hz, 9.0 Hz), 1.43 (3H) dt, 7.0 Hz, 1.0 Hz). MS (E.I.) M⁺ 368.0419. FW 368.0409.

### Synthesis of [¹⁸F]Labeling

### Fluoroethyl-4-nitrophenylphosphate

In a first method, an aqueous [¹⁸F]F⁻ solution was added to CH₃CN (0.5 mL), after which the resulting solution was evaporated under argon using an oil bath having a temperature of approximately 70° C. Acetonitrile (3×0.3 mL) was added to the resulting product and evaporated, after which a solution of ethyldi(4-nitrophenyl) phosphate (1e) (10 mg, 27 μmol) in acetonitrile (0.2 mL) was added to form a reaction mixture. The reaction mixture was heated to approximately 70° C. and maintained at this temperature for approximately 15 minutes. A sample was then withdrawn from the reaction mixture and analyzed using TLC. TLC conducted using Silica, 1% methanol-dichloromethane, Rf (1e and radiolabeled product) 0.60 indicated that the conversion of the solubilized [¹⁸F]F⁻ into the product was approximately 8%. In a second method, a solution of ethyldi(4-nitrophenyl) phosphate (1e) (5 mg, 14 μmol in acetonitrile (0.4 mL) was added to the dried [¹⁸F]F⁻ activated by KRYPTOFIX/K₂CO₃ or KRYPTOFIX/KHCO₃ to form a reaction mixture. The reaction mixture was heated to approximately 70° C. and maintained at this temperature for approximately 15 minutes. A sample was then withdrawn from the reaction mixture and analyzed using TLC as described above, but no conversion of [18F]F⁻ into the product was detected.

A summary of the P(V) precursors and the associated leaving groups and solvents as described above are reflected below in TABLE 1.

TABLE 1

| | Oxidation | Leaving | [¹⁸F]F⁻ | | Temperature | |
|---|---|---|---|---|---|---|
| | State | Group | Activator | Solvent | (° C.) | Yield^{a} |
| 1a | P(V) | Cl— | M^{b} | CH₃CN | 75 | 96% |
| 1b | P(V) | Cl— | S^{c} | DMF | 90 | 79% |
| 1c | P(V) | Cl— | S | CH₃CN | 70 | 35% |
| 1d | P(V) | MNP-^{d} | S | CH₃CN | 75 | 75% |
| 1e | P(V) | MNP- | S | CH₃CN | 70 | None |
| 1e | P(V) | MNP- | none^{e} | CH₃CN | 70 | 8% |

^{a}Conversion of the solubilized [¹⁸F]F⁻ into the product measured by TLC;
^{b}M—mild conditions, tetrabutylammonium carbonate was used to activate [¹⁸F]F⁻;
^{c}S—standard conditions, K₂CO₃/Kryptofix 222;
^{d}MNP (mono-4-nitrophenoxy) leaving group; and
^{e}No activating reagent was used during the radiofluorination process.

Each of the precursor compounds listed in TABLE 1 may be represented by a generic formula I(a) as illustrated below, with the identity of the corresponding substituents for the various precursors being reflected in TABLE 2.

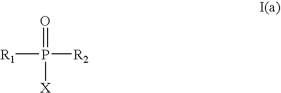

TABLE 2

| Precursor | R₁ | R₂ | X |
|---|---|---|---|
| 1a | N(CH₃)₂ | N(CH₃)₂ | Cl |
| 1b | OPh | OPh | Cl |
| 1c | OEt | OEt | Cl |
| 1d | OEt | OEt | 4-nitrophenoxy |
| 1e | OEt | 4-nitrophenoxy | 4-nitrophenoxy |

### Synthesis Of The ¹⁸F—P-Labeled Compounds From Phosphorus (III) Precursors

### Synthesis of 2,2′-Ethylidenebis(4,6-di-tert-butylphenyl) 4-nitrophenylphosphite (3g)

A solution of phosphorus trichloride (0.34 g, 2.5 mmol) in toluene (5 mL) was added to a solution of 2,2′-ethylidene-bis(4,6-di-tert-butylphenol) (1.00 g, 2.28 mmol) in toluene (5 mL) while stirring under argon. After 3 hours, a solution of 4-nitrophenol (0.32 g, 2.28 mmol) in toluene (5 mL) was added, and the mixture was allowed to sit at ambient temperature, approximately 25° C., for 2 weeks. The mixture was then filtered to remove any formed solids, and the filtrate then being evaporated and vacuum dried to obtain approximately 1.28 g of a crude product as a light gray solid. Further purification of the small fraction of the crude product (70 mg) was achieved by preparative TLC, Silica, 2.5% ethyl acetate-hexane, Rf 0.4. The desired zone of silica was eluted with 20% methanol-dichloromethane (5 mL) to obtain a solution of the desired compound, after which the solvent was evaporated to obtain approximately 22 mg (36 μmol) of a purified product 2,2′-ethylidenebis(4,6-di-tert-butylphenyl) 4-nitrophenylphosphite (3g) as a white solid. An analysis of the product yielded the following: ³¹P NMR (CDCl₃) δ 129 ppm. ¹H NMR (CDCl₃, 300 MHz) δ, 8.28 (2H, d, 9.2 Hz), 7.58 (2H, dd, 9.2 Hz, 1.1 Hz), 7.39 (2H, d, 2.4 Hz), 7.21 (2H, d, 2.3 Hz), 4.80 (1H, dd, 7.2 Hz, 2.4 Hz), 1.60 (3H, d, 7.4 Hz), 1.36 (s, 18H), 1.29 (s, 18H). ESI MS (low res.), MH⁺ 606 (FW 605.7).

### Synthesis of [¹⁸F]2,2′-Ethylidenebis(4,6-di-tert-butylphenyl) fluorophosphite (4f)

In a first method, a supernatant from the suspension of the precursor 2,2′-ethylidenebis(4,6-di-tert-butylphenyl) 4-nitrophenylphosphite (3g) (2.7 mg, 4.5 μmol) described above in acetonitrile (0.4 mL) was added to the dried [¹⁸F]F⁻ activated by tetrabutylammonium carbonate as described above to form a reaction mixture. The reaction mixture was then heated to approximately 75° C. and maintained at this temperature for approximately 7 minutes to promote nucleophilic substitution and thereby synthesize the [¹⁸F]2,2′-ethylidenebis(4,6-di-tert-butylphenyl) fluorophosphite (4f) according to the general reaction (II) as illustrated below. An analysis of the reacted mixture indicated that more than 98% of the soluble radioactivity remained in the form of unreacted [18F]F⁻.

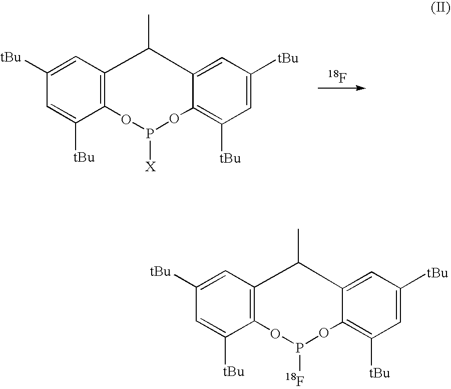

In a second method, a solution of the precursor 2,2′-ethylidenebis(4,6-di-tert-butylphenyl) 4-nitrophenylphosphite (3g) (2 mg, 3.3 μmol) in DMF (0.4 mL) was added to the dried [18F]F⁻ activated with KRYPTOFIXIK₂CO₃ to form a reaction mixture. The reaction mixture was left at room temperature, approximately 25° C., for 10 minutes to obtain the [¹⁸F]2,2′-ethylidenebis(4,6-di-tert-butylphenyl) fluorophosphite (4f). An analysis indicated that the conversion of the solubilized [¹⁸F]F⁻ into the product was 26%. TLC, 2.5% ethyl acetate-hexane, Silica, Rf (4f) 0.65; Alumina-N, Rf (4f) 0.84. HPLC, Phenomenex, Synergi, RP MAX (C12) 250×4.6 mm, 4 μm, column was run with acetonitrile at a flow rate 2 mL/min, UV 254 nm signal, t_{R} (4f) 7.4 min.

In a third method, the precursor 2,2′-ethylidenebis(4,6-di-tert-butylphenyl) fluorophosphite (3f) (10 mg, 21 μmol) was dissolved in acetonitrile (0.5 mL) and added to the dried [¹⁸F]F⁻ activated by tetrabutylammonium carbonate as described above to form a reaction mixture. The reaction mixture was heated to a temperature of approximately 85° C. and maintained at this temperature for approximately 10 minutes. An analysis of the post-reaction mixture indicated that conversion of the solubilized [¹⁸F]F⁻ into the [¹⁸F]2,2′-ethylidenebis(4,6-di-tert-butylphenyl) fluorophosphite product (4f) was approximately 26%. The product was then separated from the residual [¹⁸F]F⁻ using a Silica Sep-Pak (eluent 10% ethyl acetate-hexane, 4 mL).

A summary of the P(III) precursors and the associated leaving groups and solvents as described above are reflected below in TABLE 3.

TABLE 3

| | | | | | Tempera- | |
|---|---|---|---|---|---|---|
| Pre- | Oxidation | Leaving | [¹⁸F]F⁻ | | ture | |
| cursor | State | Group | Activator | Solvent | (° C.) | Yield^{a} |
| 3f | P(III) | F— | M | CH₃CN | 85 | 33% |
| 3g | P(III) | MNP- | M | CH₃CN | 75 | <2% |
| 3g | P(III) | MNP- | S | DMF | r.t.^{g} | 26%^{h} |

^{a}Conversion of the solubilized [¹⁸F]F⁻ into the product measured by TLC,
^{g}room temperature (approximately 25° C.),
^{h}the product of radiolabeled reaction had slightly different chromatographic properties compared to the authentic fluoride reference compound.

Each of the precursor compounds listed in TABLE 3 may be represented by a generic formula III(a) as illustrated below, with the identity of the corresponding substituents for the various precursors being reflected in TABLE 4.

TABLE 4

| | |
|---|---|
| | 111(a) |
| 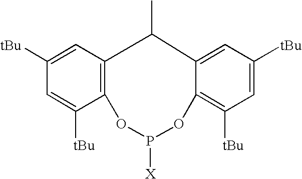 | |

| Precursor | X |
|---|---|
| 3f | ¹⁴F (unlabeled) |
| 3g | 4-nitrophenoxy |

In light of the multiple reported synthetic pathways to unlabeled phosphorofluoridates and the paucity of publications addressing ¹⁸F-radiofluorination, with the exception of the synthesis of [¹⁸F]sodium fluorophosphates as reported by Ericson in Int'l J. Appl. Radiat. Isot., 1961; 10: 177-180, the present invention may have wide applicability for producing such compounds. The exemplary examples of the disclosed method utilized simple and/or readily obtainable precursors and reference compounds so that the results could be more easily and confidently evaluated.

High specific radioactivity products (for example, a radiolabeled oligonucleotide) may be produced more efficiently by fluorination with n.c.a. [18F]F⁻. As noted above, acetonitrile or DMF are suitable reaction solvents for use in combination with [¹⁸F]F⁻ that was activated with either tetrabutylammonium carbonate (for mild 18F-fluorination), as suggested by Culbert et al. in Appl. Radiat. Isot. 1995; 46: 887-891, or KRYPTOFIX 222/K₂CO₃ complex described above for more aggressive fluorination. As illustrated above, the synthesis of phosphorofluoridates can be achieved starting with either phosphorus(III) or phosphorus(V) precursors. Generally, the P(III) precursors tend to be more reactive, and thus more easily fluorinated, the [¹⁸F] radiolabeled products manufactured from P(V) precursors tend to exhibit improved stability relative to the P(III) products. Moreover, the use of P(III) precursors typically necessitates an additional step, typically oxidation as described by Hayakawa et al. in Tetrahedron Lett. 1986; 27: 4191-4194, or sulfurization as described by Iyer et al. in J. Am. Chem. Soc. 1990; 112: 1253-1254, to bring the phosphorus to the more stable oxidation state P(V).

As described above, the radiolabeling of P(V) compounds, specifically a precursor with a P(V)-Cl bond, was demonstrated by producing a radiolabeled insecticidial compound, dimefox, in a process that converted approximately 96% of the soluble [¹⁸F]F⁻ into radiolabeled product (2a) at the room temperature. Reactions of phosphorochloridates with nucleophilic fluoride are well known as suggested by Saunders et al. in J. Chem. Soc. Abst. 1948: 695-699; Effenberger et al. in Synthesis 1981; 1: 70-72; and Gholivand et al. in Phosphorus and Sulfur 1995; 106: 173-177.

As detailed above, the fluorinating methods described above are able to radiofluorinate phosphorochloridates, such as precursors 1b and 1c. However, because P(V)-Cl-precursors can be difficult to separate from their radiolabeled products (a complication which would tend to decrease the biological specific radioactivity of the resulting radiotracer, precursors incorporating nitrophenoxy leaving groups may exhibit improved utility over precursors incorporating chloride for the production of PET radiotracers.

Although tosylate, mesylate and triflate containing precursors have been used for the formation of the P(V)—F bond as taught by Dabkowski et al. in Chem. Ber. 1982; 115: 1636-1643; Dabkowski et al. in Chem. Ber. 1985; 118: 1809-1824, compounds having nitrophenoxy leaving groups, such as 2,4-dinitrophenoxy, as taught by Johnson et al. in Nucleic Acids Res. 1975; 2: 1745-1749, and 4-nitrophenoxy, as taught by Hengge etal. in J. Am. Chem. Soc. 1995; 117: 5919-5926; Bunton etal. in Can. J Chem. 1998; 76: 946-954; and Mentz et al. in J. Chem. Soc. Perkin. Trans. 2 1995; 12: 2223-2226, tend to exhibit improved precursor stability.

As reflected above, n.c.a. radiolabeling of a precursor having a 4-nitrophenoxy leaving group, specifically 4-nitrophenyldietyl phosphate, provided a conversion of approximately 75% of the available [18F]F⁻ into the radiolabeled product. Surprisingly di(4-nitrophenyl)ethyl phosphate appeared relatively unreactive towards [18F]F⁻/KRYPTOFIX 222/K₂CO₃, a result that may possibly be attributed to precursor decomposition under the attempted synthesis conditions. However, as also demonstrated above, radiolabeling can be achieved without any [¹⁸F]F⁻ activating system.

As reflected in the discussion above, both P(V)-containing precursors and P(III)-containing precursors was investigated and successfully radiolabeled with an [¹⁸F]-P bond. Reactions of the P(III) precursors utilizing a 4-nitrophenoxy leaving group with F⁻ have been used for the synthesis of the nucleotide analogs containing phosphorofluoriditite, as discussin by Dabkowski et al. in Tetrahedron Lett. 1995; 36: 1095-1098, as well as phosphorofluoridate- and phosphorofuoridothionate moieties as taught by Dabkowski et al. in Nucleosides Nucleotides Nucleic Acids 2000; 19: 1779-1785; Dabkowski et al. J. Chem. Soc. Perkin. Trans. 1 2001: 490-493.

The remainder of the compounds used are commercially available from one or more sources such as Aldrich (2,2′-ethylidenebis(4,6-di-tert-butylphenyl) fluorophosphite) and as taught in U.S. Pat. No. RE36,128. 2,2′-ethylidenebis(4,6-di-tert-butylphenyl) fluorophosphite was utilized as a reference compound for the model reaction in light of its relatively stability (considered to be due to steric hindrance of hydrolysis by tert-butyl groups) as taught by Odorisio et al. in Phosphorus and Sulfur 1983; 15: 9-13 (“Odorisio”), the disclosure of which is incorporated herein, in its entirety, by reference. The synthesis of the 4-nitrophenoxy group containing precursor (3g) was accomplished by reaction of the precursor diol with PCl₃ and 4-nitrophenol in toluene in the presence of triethylamine (the chloride intermediate was not isolated) as taught by Odorisio; Odorisio et al. in Phosphorus and Sulfur 1984; 19: 1-10; U.S. Pat. Nos. 5,659,060 and 5,843,339, the disclosures of which are incorporated herein, in their entirety, by reference.

The reaction of precursor 3g with [¹⁸F]F⁻ resulted in formation a new radiolabeled compound (with approximately 25% conversion of the [¹⁸F]F⁻ into the product) which had close but not identical chromatographic properties with the reference compound. It is possible that the observed slight difference is the result of formation of the two isomers of the product 4f, with the radiolabeled compound having a different ratio of the isomers. Although only one conformational isomer was found for 2,2′-ethylidenebis(4,6-di-tert-butylphenyl) methylphosphite, Odorisio, because of the small size of fluorine and its ability to form hydrogen bonds, it is possible that 3f may exist as the two distinctive isomers.

Assessment of hydrolytic stability of some of the synthesized ¹⁸F-labeled compounds showed that none of them undergoes an immediate defluorination in the aqueous solution. For example, more than 75% of the n.c.a. [¹⁸F]dimefox (2a) and carrier added product 4f remained unchanged after 30 minutes of storage in aqueous media. Similarly, no decomposition of the product compound 2c was noted under similar storage conditions. It is suspected that ¹⁸F—P-bond-based PET radiotracers, possibly those containing phosphorofluoridatemonoester groups, would possess relatively greater hydrolytic stability.

No carrier added radiotracers suitable as PET scan tracers containing a ¹⁸F—P bond can be synthesized from both P(V) and P(III) containing precursors. Precursors having nitrophenoxy leaving groups attached to P(V) tend to exhibit an acceptable combination of stability and reactivity and also tend to allow the precursor compound(s) to be separated more easily from the ¹⁸F-labeled products. The method disclosed herein for producing radiolabeled compounds via the P—¹⁸F chemistry may particularly useful radiolabeling oligonucleotides, phospholipids, phosphorilated proteins, sugars and steroids for the use in PET applications.

1. A method for manufacturing ¹⁸F radiolabeled phosphorus-containing compounds comprising:
preparing an anhydrous solution of a phosphorus-containing precursor compound, the precursor compound including at least one phosphorus atom bonded to a leaving group;
introducing an activated ¹⁸F fluoride into the anhydrous solution to form a reaction mixture; and
maintaining the reaction mixture at a reaction temperature and for a reaction time sufficient to cause a first percentage of the ¹⁸F to replace the leaving group through nucleophilic substitution to form a radiolabeled compound.

2. A method for manufacturing ¹⁸F radiolabeled phosphorus-containing compounds according to claim 1, further comprising:
separating the radiolabeled compound from the reaction mixture.

3. A method for manufacturing ¹⁸F radiolabeled phosphorus-containing compounds according to claim 1, wherein:
the leaving group is selected from a group consisting of chlorine, fluorine and nitroxyphenol.

4. A method for manufacturing ¹⁸F radiolabeled phosphorus-containing compounds according to claim 1, wherein: the reaction temperature is at least 25° C. and the reaction time is at least 5 minutes.

5. A method for manufacturing ¹⁸F radiolabeled phosphorus-containing compounds according to claim 1, wherein:
the precursor compound is selected from a group consisting of nucleotides, oligonucleotides, phospholipids, phosphorilated proteins, phosphorilated sugars and steroids.

6. A method for manufacturing ¹⁸F radiolabeled phosphorus-containing compounds according to claim 2, wherein:
separating the radiolabeled compound from the reaction mixture includes the steps of thin layer chromatography (TLC) to separate compounds present in the reaction mixture within a chromatography medium; and
eluting portions of the chromatography medium to obtain a product solution containing the radiolabeled compound.

7. A method for manufacturing ¹⁸F radiolabeled phosphorus-containing compounds according to claim 1, further comprising:
preparing the activated ¹⁸F fluoride by combining an aqueous [¹⁸F]F⁻ solution with a solution of tetrabutylammonium carbonate in acetonitrile to form an activated fluoride solution; and
evaporating the solvent from the activated fluoride solution to obtain an activated ¹⁸F fluoride compound.

8. A method for manufacturing ¹⁸F radiolabeled phosphorus-containing compounds according to claim 1, further comprising:
preparing the activated ¹⁸F fluoride by combining an aqueous [¹⁸F]F⁻ solution with a solution of 4,7,13,16,21,24-hexaoxa-1,10-diazabicyclo[8.8.8]hexacosane and K₂CO₃ in a acetonitrile:water solvent to form an activated fluoride solution; and
evaporating the solvent from the activated fluoride solution to obtain an activated ¹⁸F fluoride compound.

9. A method for manufacturing 18F radiolabeled phosphorus-containing compounds according to claim 8, wherein:
the 4,7,13,16,21,24-hexaoxa-1,10-diazabicyclo[8.8.8]hexacosane and K₂CO₃ are present in the acetonitrile:water solvent in a molar ratio of from about 1:10 to about 10:1 and the acetonitrile and water are present in the solvent in a weight ratio of from about 1:3 to about 3:1.

10. A method for manufacturing ¹⁸F radiolabeled phosphorus-containing compounds comprising:
preparing an anhydrous solution of a phosphorus-containing precursor compound, the precursor compound including at least one phosphorus(III) atom bonded to a leaving group;
treating the precursor compound to produce an intermediate precursor compound in which the at least one phosphorus(III) atom is converted to a phosphorus(V) atom;
introducing an activated ¹⁸F fluoride into the anhydrous solution to form a reaction mixture; and
maintaining the reaction mixture at a reaction temperature and for a reaction time sufficient to cause a first percentage of the ¹⁸F to replace the leaving group through nucleophilic substitution to form a radiolabeled compound.
